SEQUENCE LISTING (10 sequences)

SEQ ID NO: 1 - DNA, 19 bp, synthetic construct
  source 1..19: mol_type = other DNA
ctgggctacactgagcacc

SEQ ID NO: 2 - DNA, 21 bp, synthetic construct
  source 1..21: mol_type = other DNA
aagtggtcgttgagggcaatg

SEQ ID NO: 3 - DNA, 21 bp, synthetic construct
  source 1..21: mol_type = other DNA
acgtcgaaagtattttgcgga

SEQ ID NO: 4 - DNA, 21 bp, synthetic construct
  source 1..21: mol_type = other DNA
acggttccccaggtcttaatc

SEQ ID NO: 5 - DNA, 21 bp, synthetic construct
  source 1..21: mol_type = other DNA
accacaacatgctgctccttt

SEQ ID NO: 6 - DNA, 22 bp, synthetic construct
  source 1..22: mol_type = other DNA
atcaacgtgcagccatatgtct

SEQ ID NO: 7 - DNA, 20 bp, synthetic construct
  source 1..20: mol_type = other DNA
tgtcagcgcctttgagatcg

SEQ ID NO: 8 - DNA, 21 bp, synthetic construct
  source 1..21: mol_type = other DNA
ctctggacatagggtcaggaa

SEQ ID NO: 9 - DNA, 19 bp, synthetic construct
  source 1..19: mol_type = other DNA
gggcagacggaaagtgctc

SEQ ID NO: 10 - DNA, 21 bp, synthetic construct
  source 1..21: mol_type = other DNA
ctgcacaagcatgttcagaga

(19) United States
(12) Patent Application Publication
(10) Pub. No.: US 2023/0293619 A1
(43) Pub. Date: Sep. 21, 2023

(54) METHOD FOR INCREASING MELATONIN IN SUBJECT AND REGULATING EXPRESSION OF TPH GENE, DDC GENE, AANAT GENE, AND ASMT GENE BY USING LONGAN FLOWER EXTRACT

(71) Applicant: NUTRAREX BIOTECH CO., LTD., Taichung City (TW)

(72) Inventors: Pin-Chao HUANG, Taichung City (TW); Hsi-Chi LU, Taichung City (TW)

(21) Appl. No.: 18/150,984

(22) Filed: Jan. 6, 2023

(30) Foreign Application Priority Data
Jan. 11, 2022 (TW) 111101137

(51) Int. Cl. A61K 36/77; A23L 33/105; A61K 9/00; A23L 19/00

(52) U.S. Cl. CPC A61K 36/77; A23L 19/01; A23L 33/105; A61K 9/0053

(57) ABSTRACT

The present disclosure provides a method for increasing content of melatonin in a subject and regulating expression of TPH gene, DDC gene, AANAT gene, and ASMT gene, including administering to a subject in need thereof a composition comprising an effective amount of a longan flower extract, wherein the longan flower extract can effectively increase the content of melatonin in the subject, and can directly improve sleep disturbance and insomnia, as well as the sleep quality within a few days after taking the longan flower extract. The present disclosure can help activate the melatonin synthesis pathway.

### CROSS-REFERENCE TO RELATED APPLICATION

This application claims priority of Taiwan patent application No. 111101137, filed on Jan. 11, 2022, the content of which is incorporated herein in its entirety by reference.

### REFERENCE TO AN ELECTRONIC SEQUENCE LISTING

The contents of the electronic sequence listing (2023-01-06 Sequence Listing.xml; Size: 9,740 bytes; and Date of Creation: Jan. 6, 2023) are herein incorporated by reference in its entirety.

### BACKGROUND OF THE INVENTION

### 1. Field of the Invention

The present invention relates to a method for increasing content of melatonin in a subject and regulating expression of tryptophan hydroxylase (TPH) gene, dopa decarboxylase (DDC) gene, aralkylamine N-acetyltransferase (AANAT) gene, and acetylserotonin O-methyltransferase (ASMT) gene by using a longan flower extract, especially a method for improving insomnia, sleep disturbance, or sleep quality in a subject by using the longan flower extract.

### 2. The Prior Art

Longan (Dimocarpus longans Lour.) is an evergreen tree plant of the Sapindaceae family. Taiwan is one of the major countries in the world for the production and research of longan, and longan is among the top five fruit trees in Taiwan. The production season of longan flowers is from March to April every year. Longan flowers can be divided into male flowers and female flowers. After longan flowers bloom, they will be pollinated by wind and bees. The female flowers are ready for fruit after pollination, which is not easy to fall, while the male flowers would fall naturally after about 3-5 days of flowering, can also be knocked slightly to promote falling collection to assist pollination, and does not affect the fruit yield. Male flowers that do not have the ability to bear fruit become a by-product of the longan cultivation process. Because the longan flower has a special longan aroma and a slightly sweet and astringent taste, it is often consumed in the form of flower tea or tea bag. It is rich in polyphenols and has good antioxidant, anti-inflammatory, uric acid lowering and other biological activities with development value.

Melatonin is a hormone secreted by the pineal gland of the brain and has the function of regulating the biological clock. During the day, the secretion of melatonin is inhibited by light, so the human body can stay awake. At night, the secretion of melatonin increases, allowing the human body to fall asleep. Long-term night shifts, work shifts, international jet lag during travel, mental stress, or the use of electronic products can lead to insufficient secretion of melatonin at night, resulting in poor sleep quality, and even sleep disturbances such as insomnia, as well as daytime fatigue, day and night reversal, and decreased immunity, which have a serious impact on daily life. According to the literature, in addition to regulating circadian and endocrine rhythms and helping to fall asleep, melatonin also has the ability of immune regulation, anti-oxidation, anti-inflammation, soothing irritable bowel syndrome, preventing osteoporosis and inhibiting tumors.

Currently clinically commonly used drugs for the treatment of sleep disturbances include sedative and hypnotic drugs (for example: benzodiazepine and barbiturate), and antidepressant drugs (for example: clomipramine and imipramine). However, the use of the aforementioned drugs is highly addictive and may cause symptoms such as drowsiness, nausea, headache, vomiting, gastrointestinal discomfort, memory impairment, rebound insomnia, trance, ataxia, respiratory depression, and/or sleepwalking and other side effects. At present, melatonin derived from bovine pineal gland or chemically synthesized is also sold on the market. However, due to the prevalence of mad cow disease and the uncertainty of safe dosage, these products are still strictly controlled or even banned in many countries. Therefore, it is necessary and urgent to continuously develop drugs or methods that can effectively treat insomnia, improve sleep disturbance and/or regulate sleep, and are less prone to addiction and side effects.

Melatonin can promoted by increasing the expression levels of related genes of melatonin synthesis pathway, such as tryptophan hydroxylase (TPH) gene, dopa decarboxylase (DDC) gene, aralkylamine N-acetyltransferase (AANAT) gene, and acetylserotonin O-methyltransferase (ASMT) gene

### SUMMARY OF THE INVENTION

A primary objective of the present invention is to provide a method for increasing content of melatonin in a subject, comprising administering to a subject in need thereof a composition comprising an effective amount of a longan flower extract, wherein the longan flower extract is obtained by extracting a longan with a solvent, and the solvent is water, alcohol, a mixture of alcohol and water, or a combination thereof.

Another objective of the present invention is to provide a method for regulating expression of tryptophan hydroxylase (TPH) gene, dopa decarboxylase (DDC) gene, aralkylamine N-acetyltransferase (AANAT) gene, and acetylserotonin O-methyltransferase (ASMT) gene, comprising administering to a subject in need thereof a composition comprising an effective amount of a longan flower extract, wherein the longan flower extract is obtained by extracting a longan with a solvent, and the solvent is water, alcohol, a mixture of alcohol and water, or a combination thereof.

According to an embodiment of the present invention, the longan flower extract improves insomnia, sleep disturbance, or sleep quality in the subject.According to an embodiment of the present invention, the composition comprises at least 0.2 wt% of the longan flower extract.

According to an embodiment of the present invention, the longan is further hydrolyzed with a complex cellulase before being extracted with the solvent.

According to an embodiment of the present invention, the composition is a pharmaceutical composition or a food composition.

According to an embodiment of the present invention, the composition further comprises a pharmaceutically acceptable carrier or an edible material.

According to an embodiment of the present invention, the composition is in a form of a powder, a granule, a solution, a gel, or a paste.

According to an embodiment of the present invention, the TPH gene is TPH1 gene.

According to an embodiment of the present invention, the effective amount of the longan flower extract is 25 µg/mL to 50 µg/mL.

The longan flower extract of the present invention has the effect of increasing content of melatonin in the subject, and can directly improve sleep disturbance and insomnia, as well as the sleep quality within a few days after taking the longan flower extract. Therefore, the longan flower extract of the present invention can be used to prepare a composition for helping sleep and/or improving sleep quality, wherein the composition is a medicament, a food, or a skin care product, and can be administered to a subject by oral administration, skin application, etc.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows the result of the effect of the longan flower extract of the present invention to increase the content of melatonin in a subject.

FIG. 2 shows the result of the effect of the longan flower extract of the present invention on improving sleep disturbance in a subject.

FIG. 3 shows the result of the effect of the longan flower extract of the present invention on improving the sleep quality in a subject.

FIG. 4 shows that the longan flower extract of the present invention has the effect on modulating the expression of tryptophan hydroxylase 1 (TPH1) gene, dopa decarboxylase (DDC) gene, aralkylamine N-acetyltransferase (AANAT) gene, and acetylserotonin O-methyltransferase (ASMT) gene.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

As used herein, the data provided represent experimental values that can vary within a range of ±20%, preferably within ±10%, and most preferably within ±5%.

Statistical analysis is performed using the Excel software. Data are presented as mean ± standard deviation (SD), and differences between them are analyzed by the student’s t-test.

Unless otherwise stated in the context, the terms “a”, “the” and similar terms used in the present specification (especially in the claims of the patent application described later) should be understood to include both singular and plural forms. The term “treatment” should not be interpreted as treating a subject until complete recovery, but should include maintaining a subject’s disease progression or symptoms to a substantially static level, increasing a subject’s rate of recovery, improving the severity of a particular condition, or improving a patient’s quality of life. The term “regulation” refers to positive regulation (including induction, stimulation, and enhancement) or negative regulation (including inhibition and reduction) to move a subject toward a normal state of the physiology. The term “subject” means a human or non-human mammal.

The term “longan flower extract” described herein refers to the product obtained by extracting longan by a solvent for a specific time and temperature.

The term “sleep disturbance” described herein includes, but is not limited to, difficulty falling asleep, light sleep, insufficient sleep depth, easy waking up, inability to maintain longer sleep, and quality of life affected by sleep.

The longan flower extract used in the present invention can be provided by an operation comprising the following steps: (a) extracting longan flowers with an extraction solvent to provide a crude extract; (b) centrifuging the crude extract, and the supernatant is filtered to provide a filtrate; (c) concentrating the filtrate under reduced pressure to obtain a concentrated extract; and (d) drying the concentrated extract to obtain a dried product.

In addition, as needed, before or when step (a) is performed, other operations such as heating, cooling, stirring, ultrasonic waves, etc., are supplemented to improve the extraction effect. For example, in an embodiment of the present invention, the stirring operation is performed before the step (a) is performed.

In addition, in step (c), the temperature can be adjusted when concentrating under reduced pressure as needed. For example, the concentration under reduced pressure of step (c) can be carried out at 45-70° C. In an embodiment of the present invention, the concentration under reduced pressure of step (c) is carried out at 55±5° C. The longan flower extract used according to the present invention can be either the concentrated extract provided in step (c) or the dried product provided in step (d), wherein, step (d) can be carried out by freeze drying, spray drying and the like. In order to achieve the maximum extraction benefit as much as possible, before performing step (b), the longan flower can be repeatedly extracted with the same or different extraction solvent, and the extracts obtained from the multiple extractions can be combined to provide the crude extract for step (b). The cycle of step (b), step (c), and other operations as required can also be repeated.

The composition provided according to the present invention can be a pharmaceutical composition or a food composition. The pharmaceutical composition provided according to the present invention can be used for at least one of the following: increasing the content of melatonin, improving or treating insomnia, improving sleep disturbance, or improving sleep quality. Preferably, it is used to increase the content of melatonin. The food composition provided according to the present invention is used for at least one of the following: increasing the content of melatonin, improving or treating insomnia, improving sleep disturbance, or improving sleep quality. Preferably, it is used to increase the content of melatonin.

The pharmaceutical composition provided according to the present invention can be a medicament, a nutritional supplement, a health food, or any combination thereof, and can further comprise a pharmaceutically acceptable excipient, vehicle, adjuvants, and/or food additives.

The pharmaceutical composition provided according to the present invention can be in any suitable form, without special limitations, and is in a corresponding appropriate dosage form depending on the intended use. For example, without limitation, the pharmaceutical composition can be administered to a subject in need thereof by oral or parenteral (e.g., transdermal) administration.

In a preferred embodiment of the present invention, the composition of the present invention is formulated in a pharmaceutically acceptable vehicle and made into a dosage form suitable for oral administration, injection, and the pharmaceutical composition is preferably in a dosage form selected from the group consisting of: solution, suspension, powder, tablet, pill, syrup, lozenge, troche, chewing gum, capsule, and the like.

According to the present invention, the pharmaceutically acceptable vehicle can comprise one or more reagents selected from the group consisting of solvent, buffer, emulsifier, suspending agent, decomposer, disintegrating agent, dispersing agent, binding agent, excipient, stabilizing agent, chelating agent, diluent, gelling agent, preservative, wetting agent, lubricant, absorption delaying agent, liposome, and the like. The selection and quantity of these reagents fall within the scope of the professional literacy and routine techniques of those skilled in the art.

According to the present invention, the pharmaceutically acceptable vehicle comprises a solvent selected from the group consisting of water, normal saline, phosphate buffered saline (PBS), aqueous solution containing alcohol, and combinations thereof.

Taking the dosage form suitable for transdermal administration as an example, the pharmaceutical composition provided according to the present invention can be in the form of patches, lotions, creams, gels (for example: hydrogels), pastes (for example: dispersion paste, ointment), spray, or solution (for example: suspension), etc., but not limited to this.

The pharmaceutical composition provided according to the present invention can be administered with different administration frequencies such as once a day, multiple times a day, or once a few days, depending on the individual’s needs, age, weight and health status. In the pharmaceutical composition provided according to the present invention, the content ratio of the longan flower extract in the composition can be adjusted according to actual application requirements. In addition, the pharmaceutical composition may additionally contain one or more other active ingredients (such as sleeping drugs, antidepressants, and melatonin, etc.) as needed, or be used in combination with drugs containing the one or more other active ingredients in order to further strengthen the effect of the pharmaceutical composition or increase the flexibility and deployment degree of the formulation, as long as the other active ingredients have no adverse effect on the benefits of the active ingredients of the present invention (i.e., the longan flower extract).

Optionally, the pharmaceutical composition or food composition provided according to the present invention may additionally contain an appropriate amount of additives, such as flavoring agents, toners, colorants, etc. that can improve the taste and visual perception of the pharmaceutical composition or food composition when taking it, and buffers, save agents, preservatives, antibacterial agents, antifungal agents, etc. that can improve the stability and storage of the pharmaceutical composition or food composition.

The food composition provided by the present invention can be a food product, and is formulated with edible materials to include but not limited to: beverages, fermented foods, bakery products, health foods, nutritional supplements, and dietary supplements.

According to the present invention, the edible material is selected from the group consisting of: water, fluid milk products, milk, concentrated milk; fermented milk such as yogurt, sour milk, frozen yogurt, and lactic acid bacteria-fermented beverages; milk powder; ice cream; cream cheeses; dry cheeses; soybean milk; fermented soybean milk; vegetable-fruit juices; juices; sports drinks; confectionery; jellies; candies; infant formulas; health foods; animal feeds; Chinese herbals; and dietary supplements.

According to the present invention, the food product can be regarded as a food additive, which is added during the preparation of raw materials by conventional methods, or added in the production process of food, and formulated with any edible material into food products for human and non-human animals to eat.

The beverage, fermented food, bakery product, health food, nutritional supplements, and dietary supplements provided according to the present invention can be eaten with different frequencies such as once a day, multiple times a day, or once a few days, depending on the individual’s age, weight and health status. The content of the longan flower extract in the beverage, fermented food, bakery product, health food, nutritional supplement and dietary supplement provided according to the present invention can also be adjusted according to the needs of specific groups, for example, adjusted to be taken every day amount.

For beverages, fermented foods, bakery products, health foods, nutritional supplements and/or dietary supplements provided according to the present invention, the recommended usage amount can be marked on their outer packaging, the usage standards and conditions for specific groups (for example: insomniacs, depression patients, pregnant woman, etc.), or recommendations for co-administration with other foods or medicines, etc., to facilitate users to take them without the guidance of doctors, pharmacists or relevant deacons without safety concerns. In the food composition provided according to the present invention, the aspects of the longan flower extract and related applications are as described above.

### Example 1

### Preparation Process of Longan Flower Extract of Present Invention

In one embodiment of the present invention, the dried longan flower and the extraction solvent such as water, alcohol, or mixture of alcohol and water were uniformly mixed with a volume ratio of 1-5:5-20, and then extraction was carried out at 50-100° C. for 30-60 minutes to obtain crude extract, wherein the extraction solvent is preferably water or alcohol and more preferably ethanol. After the crude extract was cooled to room temperature, it was centrifuged at 1,000 g for 10 minutes and the supernatant was collected. The supernatant was filtered through a 400-mesh filter to obtain a filtrate. Next, the filtrate was concentrated under reduced pressure at 45-70° C. to obtain the longan flower extract of the present invention in the form of a concentrated solution, and then spray drying can be carried out to obtain the dry form of the longan flower extract of the present invention.

In a preferred embodiment of the present invention, the enzyme hydrolysis step can be selectively carried out before the extraction step. Specifically, after adding the extraction solvent to the dried longan flowers, 0.05-0.2% (w/w) complex cellulase was additionally added for 30-60 minutes, and extraction was carried out at 50-100° C. for 30-60 minutes. The complex cellulase can be Viscozyme® (Sigma, containing β-dextranase, Xylanase, Cellulase, and Hemicellulase), or it can be Celluclast® (Sigma, activity ≥700 units/g, containing Cellulase). The enzymatic hydrolysis step can improve the yield and active ingredient content of the longan flower extract of the present invention.

In the following examples, water was used as the extraction solvent for extracting the longan flower extract, and was uniformly mixed at a volume ratio of 1:10-20, and extraction was carried out at 75-85° C. for 30-60 minutes to obtain crude extract. After cooling to room temperature, centrifugation was performed at 1,000 g for 10 minutes and the supernatant was collected. After filtering through a 400 mesh filter screen, the filtrate was concentrated under reduced pressure at 55-60° C., and then spray drying was carried out to obtain the longan flower extract of the present invention.

### Example 2

### Longan Flower Extract of Present Invention Increases Content of Melatonin in Individuals

Melatonin is known to be an important hormone in regulating the biological clock of an individual, which can help sleep and regulate metabolism. Therefore, in one embodiment of the present invention, in order to confirm the effect of the longan flower extract of the present invention on promoting the production of melatonin in an individual, the longan flower extract of the present invention prepared by the above-mentioned preparation process is firstly obtained. 5 subjects aged 25-40 who were judged to have sleep disorders were recruited after assessment by the Athens Insomnia Scale and the Pittsburgh Sleep Quality Index which would be described later. For the first three days of the trial, the subjects were stopped from taking drugs and related health food that can affect sleep. 150-200 mL of warm water was used to brew 0.4 g of the longan flower extract of the present invention in a dry form (conversion mass percentage concentration is 0.2 wt%-0.267 wt%), and these subjects were made to take it once 1 hour before going to bed, and so on for a total of 28 days. On the 0th day, the 3rd day and the 28th day of the experiment, the saliva of each subject was sampled at the same time in the evening to detect the content of melatonin in saliva. It is necessary to stop eating for 30 minutes before sampling, and rinse the mouth to empty the food residues in the mouth. Before sampling, all the saliva remaining in the mouth was swallowed, about 1 mL of saliva under the tongue was collected with a plastic dropper, and stored in a test tube at -20° C. These collected samples were analyzed with the LDN® Melatonin Direct Saliva ELISA kit.

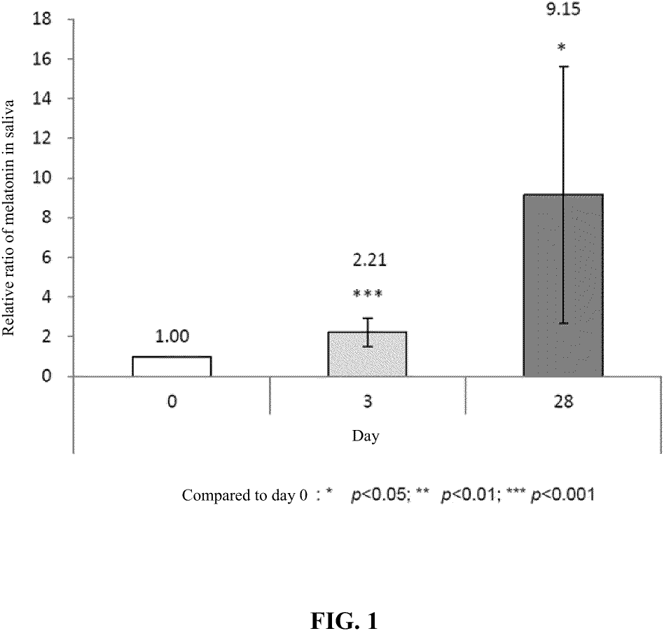

The effect of the longan flower extract of the present invention to improve the content of melatonin in individuals is shown in FIG. 1. Compared with before taking the longan flower extract of the present invention (i.e.,, the 0th day), the melatonin content in the saliva of the subjects at night can be significantly increased by 2.2 times on the 3rd day after taking the longan flower extract. On the 28th day after taking the longan flower extract, the melatonin content is significantly increased by 9.15 times. The result shows that the longan flower extract of the present invention can effectively increase the content of melatonin in individuals, so as to achieve the effects of helping sleep and regulating metabolism, and can play the function in a short period of time. The longan flower extract of the present invention may regulate the expression of melatonin-related genes in individuals, such as human tryptophan hydroxylase isoform (TPH) gene, arylalkylamine N-acetyltransferase (AANAT) gene, acetylserotonin methyltransferase (ASMT) gene, and dopa decarboxylase (DDC) gene, thereby increasing the level of melatonin in individuals, and having the effect of regulating melatonin synthesis pathway.

### Example 3

### Longan Flower Extract of Present Invention Improves Insomnia and Sleep Quality in Individuals

In one embodiment of the present invention, in order to confirm the effect of the longan flower extract of the present invention on directly improving insomnia and sleep quality in individuals, firstly, the longan flower extract of the present invention is obtained by the aforementioned preparation process. The longan flower extract was taken by the subjects according to the method described in Example 2, and then the Athens Insomnia Scale and the Pittsburgh Sleep Quality Index were evaluated on the 0th day, the 3rd day and the 28th day of the experiment respectively.

The Athens Insomnia Scale is an assessment method developed in 2000 by a group of researchers from Athens, Greece to evaluate sleep disorders in patients with insomnia. After the subjects have filled out all the scores, all the scores are added up, and the total score is 24. A score of 4-5 indicates latent insomnia; a score of ≥6 indicates sleep disturbance with insomnia.

The Pittsburgh sleep quality index (PSQI) is a method established by Buysse et al. of the University of Pittsburgh in 1989 to assess the sleep quality of patients with sleep disorders, mental disorders and the general public. Seven indicators including sleep quality, sleep lag period, total sleep hours, habitual sleep efficiency, sleep disturbance (such as insomnia), use of sleeping pills and daytime dysfunction. In addition, it also includes evaluation factors such as whether sleep is interrupted due to physiological factors such as cough and dryness and heat. After the subjects have filled in, all the scores would be added up. If the score is greater than or equal to 5 points, it can be identified as having a sleep quality disorder. The higher the score, the worse the sleep quality.

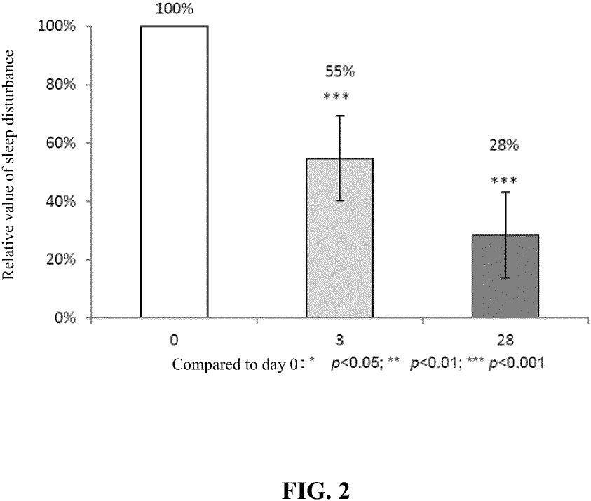

The effect of the longan flower extract of the present invention to improve sleep disturbance in individuals is shown in FIG. 2. It is calculated by self-control, and the statistical formula is: relative value of sleep disturbance (%)=[points after using the longan flower extract of the present invention/points before using the longan flower extract of the present invention] * 100%. Compared with before taking the longan flower extract of the present invention (i.e., day 0), the sleep disturbance of the subjects can be significantly reduced by 47% on the 3rd day after taking it. On the 28th day after taking it, sleep disturbance is significantly reduced by 69%. This result shows that the longan flower extract of the present invention can effectively improve insomnia problem in individuals, and can play the function in a short period of time.

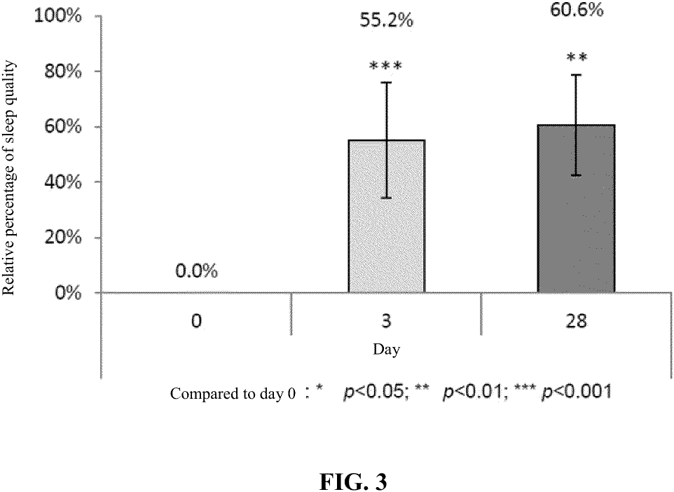

The effect of the longan flower extract of the present invention to improve sleep quality in individuals is shown in FIG. 3. It is calculated by self-control, and the statistical formula is: relative percentage of sleep quality (%)=1-[points after using the longan flower extract of the present invention/points before using the longan flower extract of the present invention] * 100%. Compared with before taking the longan flower extract of the present invention (i.e., day 0), the sleep quality of the subjects can be significantly increased by 55.2% on the 3rd day after taking it. On the 28th day after taking it, sleep quality is significantly increased by 60.6%. This result shows that the longan flower extract of the present invention can effectively improve sleep quality in individuals, and can play the function in a short period of time.

### Example 4

### Evaluation of Effect of Longan Flower Extract of Present Invention on Modulating Expression of Tryptophan Hydroxylase 1 (Tph1) Gene, Dopa Decarboxylase (Ddc) Gene, Aralkylamine N-Acetyltransferase (Aanat) Gene, And Acetylserotonin O-Methyltransferase (Asmt) Gene

The sample used in this example is powder of the longan flower extract of the present invention. The cell line used is human neuroblastoma cell line SH-SY5Y, purchased from American Type Culture Collection (ATCC). The cell culture-related consumables, reagents, their corresponding manufacturers and catalog numbers used in this example are shown in Table 1.

TABLE 1

| consumables and reagents | manufacturers | Catalog No. |
|---|---|---|
| Dulbecco’s Modified Eagle Medium | Gibco™, USA | 12100046 |
| Ham’s F-12 Nutrient Mix | Gibco™, USA | 21700075 |
| Fetal Bovine Serum (FBS) | Gibco™, USA | 10437028 |
| Trypsin-EDTA | Gibco™, USA | 16170060 |
| 0.22 µm filter cup | Millipore, Germany | S2GPT05RE |
| Trypan Blue stain | Gibco™, USA | T10282 |
| Sodium hydrogen carbonate | Panreac, Spain | 144-55-8 |

The quantitative polymerase chain reaction (qPCR) related reagents used in this example and their corresponding manufacturers and catalog numbers are shown in Table 2.

TABLE 2

| reagents | manufacturers | Catalog No. |
|---|---|---|
| iScript™ cDNA Synthesis Kit | BIO-RAD, USA | 1708891 |
| Total RNA Purification Kit | GeneMark, Taiwan | TR01-150 |
| iQ™ SYBR® Green Supermix | BIO-RAD, USA | 1708882 |
| ROX Reference Dye | Invitrogen™, USA | 12223012 |

The corresponding genes and nucleotide sequences of the primers used in this example are shown in Table 3.

TABLE 3

| | | |
|---|---|---|
| Genes | Primer forward sequence | Primer reverse sequence |
| GAPDH | CTGGGCTACACTGAGCAC C (SEQ ID NO: 1) | AAGTGGTCGTTGAGG GCAATG (SEQ ID NO:2) |
| TPH1 | ACGTCGAAAGTATTTTGCG GA (SEQ ID NO:3) | ACGGTTCCCCAGGTCT TAATC (SEQ ID NO:4) |
| DDC | ACCACAACATGCTGCTCCT TT (SEQ ID NO:5) | ATCAACGTGCAGCCAT ATGTCT (SEQ ID NO:6) |
| AANAT | TGTCAGCGCCTTTGAGATC G (SEQ ID NO:7) | CTCTGGACATAGGGTC AGGAA (SEQ ID NO:8) |
| ASMT | GGGCAGACGGAAAGTGCT C (SEQ ID NO:9) | CTGCACAAGCATGTTC AGAGA (SEQ ID NO:10) |

The procedure for cell differentiation is as follows. After SH-SY5Y cells had grown to a sufficient number, the cells were evenly plated into a 6-well culture dish at 2.5 × 10⁵ cells/well, and allowed to stand for a day to make the cells flat on the bottom of the culture dish. 10 µM retinoic acid (RA) containing 3% FBS was used as a differentiation reagent for 6 days of differentiation, and the medium was changed every two days during the differentiation period.

The procedure of the RNA extraction is as follows. After SH-SY5Y cells were differentiated under the above differentiation conditions for 6 days, 50 ppm of the longan flower extract was added to culture for 18 hours, the medium was removed, and the cells were rinsed with PBS. RNA was extracted using a commercial total RNA purification kit. 350 µl of RNA/2-mercaptoethanol (2-ME) solution was added, cells were scraped with a cell scraper and the cell fluid was collected to the RNA spin column. The filtrate was removed by centrifugation at 13200 rpm for 1 min. 500 µl of RNA Wash Solution I was added, and the filtrate was removed by centrifugation at 13200 rpm for 1 min. 82 µl of DNase I solution (80 µl DNase I incubation buffer + 2 µl DNase I) was added and reacting was performed at room temperature for 15 minutes, 500 µl of RNA Wash Solution I was added, and the filtrate was removed by centrifugation at 13200 rpm for 1 min. 600 µl of RNA Wash Solution II was added, and the filtrate was removed by centrifugation at 13200 rpm for 1 min, repeated once. Centrifugation was performed in an empty tube at 13200 rpm for 3 minutes to remove residual alcohol. The RNA spin column was put into a new 1.5 ml microcentrifuge tube, 30 µl of nuclease-free water was added for 1 minute, centrifugation was performed at 13200 rpm for 1 minute to obtain RNA, and stored at -80° C. for later use.

The procedure of reverse transcription is as follows. The RNA obtained above was quantified using an ultra-trace nucleic acid quantitative spectrometer, and then reverse transcribed with a commercial iScript™ cDNA Synthesis Kit (Bio-Rad). 20 ng RNA was added to 4 µl of 5x iScript Reaction Mix, 1 µl of iScript Reverse Transcriptase and nuclease-free water to a total volume of 20 µl. Reverse transcription was performed under the conditions listed in Table 4.

TABLE 4

| | |
|---|---|
| Priming | 5 min at 25° C. |
| Reverse transcription | 20 min at 46° C. |
| RT inactivation | 1 min at 95° C. |
| Optional step | Hold at 4° C. |

The reaction components and addition amounts of real-time PCR are shown in Table 5, and the thermal program is shown in Table 6.

TABLE 5

| | |
|---|---|
| 2x SYBR Master Mix | 10µl |
| Forward Primer | 0.5µl |
| Reverse Primer | 0.5µl |
| Water | 6.6µl |
| ROX Reference Dye | 0.4µl |
| cDNA | 2µl |
| Total | 20µl |

TABLE 6

| | |
|---|---|
| 95° C. | 10:00 min |
| 95° C. | 00:15 min |
| 60° C. | 01:00 min |
| 95° C. | 00:15 min |
| 60° C. | 01:00 min |
| 95° C. | 00:15 min |

Relative gene quantification was performed using the quantitative-comparative Ct (ΔΔCt) method. ΔCt = Ct ₛₐₘₚₗₑ ₒᵣ _{control} - Ct _{GAPDH}; ΔΔCt = ΔCt ₛₐₘₚₗₑ -ΔCt _{control}; Gene expression Relative Quantity = 2^{-ΔΔCt mean}.

The regulatory genes involved in the synthesis pathway of melatonin include TPH1, DDC, AANAT and ASMT. The body converts tryptophan many times to finally generate melatonin. However, with the increase of age or the influence of life stress, the ability of melatonin synthesis pathway may be reduced, resulting in insufficient production of melatonin and related symptoms such as sleep disorders. By increasing the expression of TPH1, DDC, AANAT and ASMT genes, it can help activate the melatonin synthesis pathway in the body, promote the normal secretion of melatonin, and reduce the dependence on drugs.

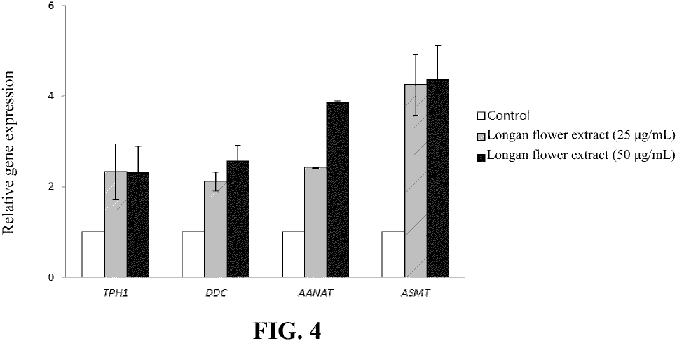

The result of this example is shown in FIG. 4. The experimental result shows that the longan flower extract at 25 and 50 µg/mL can effectively increase the expression of the key genes TPH1, DDC, AANAT and ASMT genes in the synthesis pathway of melatonin.

In summary, the longan flower extract of the present invention has the effect of increasing content of melatonin in individuals, and can directly improve sleep disturbance and insomnia, as well as the sleep quality within a few days after taking the longan flower extract. Therefore, the longan flower extract of the present invention can be used to prepare a composition for helping sleep and/or improving sleep quality, wherein the composition is a medicament, a food, or a skin care product, and can be administered to an individual by oral administration, skin application, etc.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for increasing content of melatonin in a subject, comprising administering to the subject in need thereof a composition comprising an effective amount of a longan flower extract, wherein the longan flower extract is obtained by extracting a longan flower with a solvent, and the solvent is water, alcohol, a mixture of alcohol and water, or a combination thereof.

2. The method according to claim 1, wherein the composition comprises at least 0.2 wt% of the longan flower extract.

3. The method according to claim 1, wherein the longan is further hydrolyzed with a complex cellulase before being extracted with the solvent.

4. The method according to claim 1, wherein the longan flower extract improves insomnia, sleep disturbance, or sleep quality in the subject.

5. The method according to claim 1, wherein the composition is a pharmaceutical composition or a food composition.

6. The method according to claim 1, wherein the composition further comprises a pharmaceutically acceptable carrier or an edible material.

7. The method according to claim 1, wherein the composition is in a form of a powder, a granule, a solution, a gel, or a paste.

8. A method for regulating expression of tryptophan hydroxylase (TPH) gene, dopa decarboxylase (DDC) gene, aralkylamine N-acetyltransferase (AANAT) gene, and acetylserotonin O-methyltransferase (ASMT) gene, comprising administering to a subject in need thereof a composition comprising an effective amount of a longan flower extract, wherein the longan flower extract is obtained by extracting a longan with a solvent, and the solvent is water, alcohol, a mixture of alcohol and water, or a combination thereof.

9. The method according to claim 8, wherein the longan flower extract is selected from the group consisting of longan flower extract.

10. The method according to claim 8, wherein the TPH gene is TPH1 gene.

11. The method according to claim 9, wherein the effective amount of the longan flower extract is 25 µg/mL to 50 µg/mL.

12. The method according to claim 8, wherein the longan is further hydrolyzed with a complex cellulase before being extracted with the solvent.

13. The method according to claim 8, wherein the composition is a pharmaceutical composition or a food composition.

14. The method according to claim 8, wherein the composition further comprises a pharmaceutically acceptable carrier or an edible material.

15. The method according to claim 8, wherein the composition is in a form of a powder, a granule, a solution, a gel, or a paste.
